(19) United States
(12) Patent Application Publication
(10) Pub. No.: US 2004/0143219 A1
(43) Pub. Date: Jul. 22, 2004

(54) Apparatus for inserting guide wire for use in a catheter

(76) Inventors: Gil-Whan Lee, Seoul (KR); Seung-Taek Oh, Seoul (KR); Chung-Su Kim, Seoul (KR); Ki-Jun Kim, Seoul (KR); Kyung Sun, Seoul (KR)

Correspondence Address: ST. ONGE STEWARD JOHNSTON & REENS, LLC, 986 BEDFORD STREET, STAMFORD, CT, 06905-5619, US

(21) Appl. No.: 10/747,334

(22) Filed: Dec. 29, 2003

(30) Foreign Application Priority Data
Jun. 28, 2001 (KR) KR 2001-37403

(51) Int. Cl. A61M 5/178

(52) U.S. Cl. 604/167.03; 600/585

(57) ABSTRACT

A guide wire insertion device for a catheter includes a needle portion provided with a hollow portion, the needle portion having a needle mounted on a front end; a valve mounted in the hollow portion of the needle portion to control flow of blood, a cap portion mounted on a rear end of the hollow portion of the needle portion to fix the valve means in the needle portion, and a syringe for absorbing the blood through the needle portion by penetrating the valve means.

This application is a continuation of pending International Patent Application No. PCT/KR02/01173 filed on Jun. 20, 2002 which designates the United States and claims priority of pending Korean Application No. 2001-37403 filed on Jun. 28, 2001.

### FIELD OF THE INVENTION

The present invention relates to a guide wire insertion device for a catheter and, more particularly, to a guide wire insertion device having a check valve, for preventing blood from being gush out through a needle inserted in the body.

### DESCRIPTION OF THE RELATED ART

Generally, a medical catheter is a hollow tube for insertion into a body cavity for diagnostic purposes, medicine injection or distending a contracted passageway or treatment purposes including fluid infusion, drug injection, and dilation of stenotic tracts. Such a catheter is used to treat diseases relating to a variety of internal organs such as heart, duodenum and the like. Particularly, catheter is used to treat disease of a blood vessel such as artery and vein.

The catheter can be inserted into the body either by a surgical cut down technique or by the Seldinger technique. In the Seldinger technique, a catheter can be inserted less invasively by using a guide wire.

FIGS. 1(a) and 1(b) show a process for inserting the catheter into the human body. As shown in the drawings, to insert the catheter into the body, a guide wire insertion device for the catheter is first assembled. That is, a syringe 2 is mounted on a rear end of a needle portion 1. At this point, since a piston 7 of the syringe 2 is in a state pulled rearward, an internal pressure of the syringe 2 becomes lower than the atmosphere pressure.

In this state, an operator inserts ‘the guide wire insertion device for a catheter’ into the human body so that the needle 4 can be advanced into a blood vessel.

When the needle 4 is advanced into the blood vessel, as the internal pressure of the blood vessel is higher than the internal pressure of the syringe 2, the blood flows into the syringe 2 through the needle 4. Accordingly, the operator can confirm the accurate insertion position of the needle by observing the blood flowing into the syringe 2.

After identifying the blood flowing into the syringe 2, the syringe 2 is separated from the rear end of the needle 1 to insert the guide wire 6. At this point, the blood in the needle portion 1 is gushed out.

Accordingly, the administrator blocks the rear end of the needle portion 1 using his/her finger to prevent the gush out of the blood.

After the above, the administrator prepares a guide wire 6 and removes the finger from the rear end 3 of the needle portion 1, after which the guide wire 6 is inserted in the blood vessel through a hollow hole 8 of the needle portion 1, and the guide wire 6 is placed into the desired portion of the blood vessel.

Although the above procedures have been a standard in catheter insertion, there are several limitations.

However, it is troublesome for an operator to block the rear end of the needle using the finger for preventing gush out of the blood during the procedures. As it often makes the needle position unstable, the sharp end of the needle tends to damage the vessel wall.

Furthermore, there may be sometimes possibility that the needle portion is not completely blocked, which results on gush out of blood and contamination.

In addition, the blood may be gushed out through the needle portion even during the process for inserting the guide wire.

Furthermore, of the guide wire insertion devices, one system allows a guide wire to be inserted through the rear end of the syringe without separation from the needle. However, it is sometimes difficult for an operator to handle because its wide range of hand motion and long working distance.

### SUMMARY OF THE INVENTION

Therefore, the present invention has been made in an effort to solve the above-described problems. It is an objective of the present invention to provide a guide wire insertion device for a catheter, which can prevent blood from being gush out when a needle is inserted into a blood vessel, thereby sanitarily inserting the guide wire into the blood vessel.

To achieve the above objective, the present invention provides a guide wire insertion device for a catheter, comprising a needle portion provided with a hollow portion, the needle portion having a needle mounted on a front end; valve means mounted in the hollow portion of the needle portion to control flow of blood; a cap portion mounted on a rear end of the hollow portion of the needle portion to fix the valve means in the needle portion; and a syringe for absorbing the blood through the needle portion by penetrating the valve means.

According to a preferred embodiment of the present invention, the valve means comprises a check valve for controlling the flow of the blood, a rod portion mounted on a rear portion of the check valve to open/close the check valve, and a spring for biasing the rod rearward to prevent the check valve from being opened when the syringe is removed by pushing the rod portion rearward.

The check valve comprises a circular flange having a central circular hole and a hemispherical portion projected from a surface of the circular flange and facing the needle, the hemispherical portion being provided at its top with a cut-away portion and a hollow portion communicating with the circular hole of the circular flange.

The check valve is designed such that the cut-away portion is elastically closed by self-elastic force.

The rod portion is formed in a hollow tube, one end of the rod portion has a diameter less than that of the circular hole such that the one end can be advanced into the hemispherical portion through the circular hole, wherein when the syringe is pushed forward, a front end of the syringe pushes the rod portion forward to open the cut-away portion.

One end of the spring is supported on the circular flange of the check valve and the other end of the spring is supported on a middle step formed on a middle portion of the rod portion.

The cap portion is provided with a hollow portion in which the rod portion is reciprocally mounted, the cap portion being fixed on the needle portion by a hook step formed on a front end of the cap portion and coupled to a hook groove formed on an inner circumference of the needle portion.

### BRIEF DESCRIPTION OF DRAWINGS

FIGS. 1(a) and 1(b) are views illustrating a method for inserting a guide wire into a blood vessel using a conventional guide wire insertion device for a catheter;

FIG. 2 is an exploded perspective view of a guide wire insertion device for a catheter according to a preferred embodiment of the present invention;

FIG. 3 is a sectional view of a guide wire insertion device depicted in FIG. 2;

FIG. 4 is an exploded perspective view illustrating an internal structure of a guide wire insertion device depicted in FIG. 2;

FIG. 5 is a perspective view of a cut-away portion formed in front of a valve depicted in FIG. 4; and

FIGS. 6(a) and 6(b) are views illustrating a method for inserting a guide wire into a blood vessel using a guide wire insertion device for a catheter according to a preferred embodiment of the present invention.

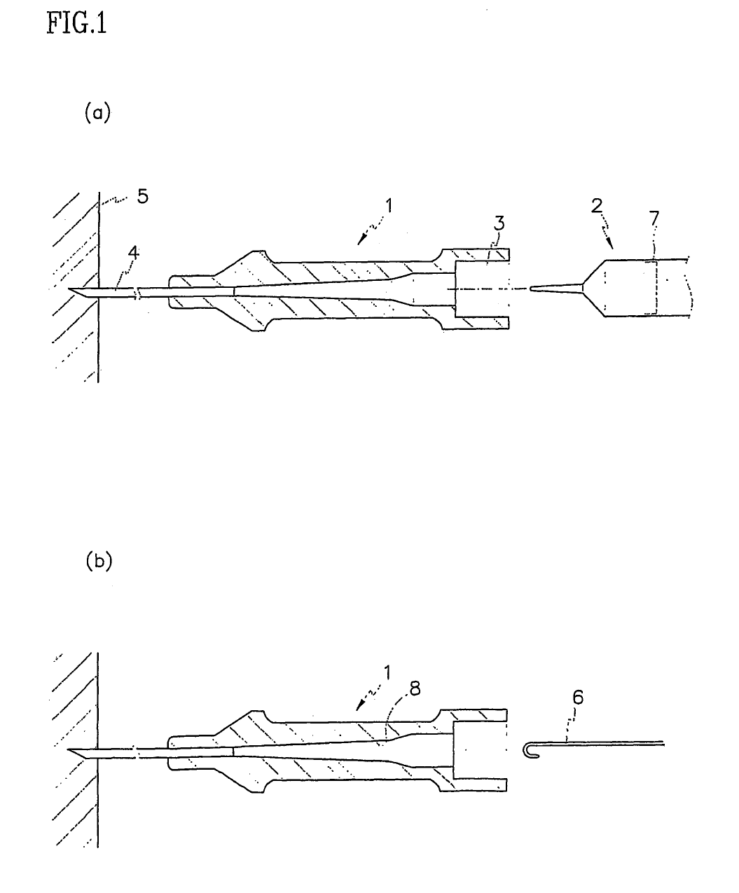

### DETAILED DESCRIPTION OF PREFERRED EMBODIMENTS

A preferred embodiment of the present invention will be described more in detail hereinafter in conjunction with the accompanying drawings.

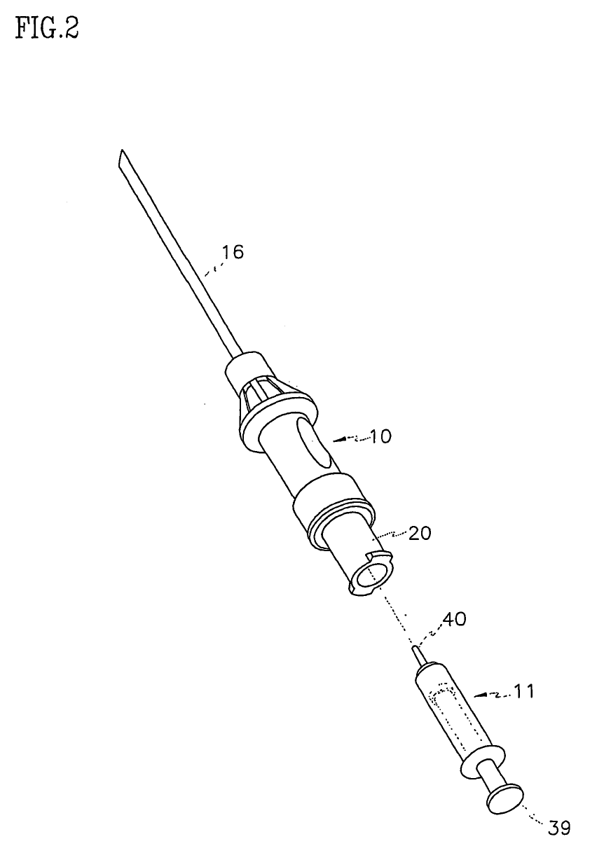

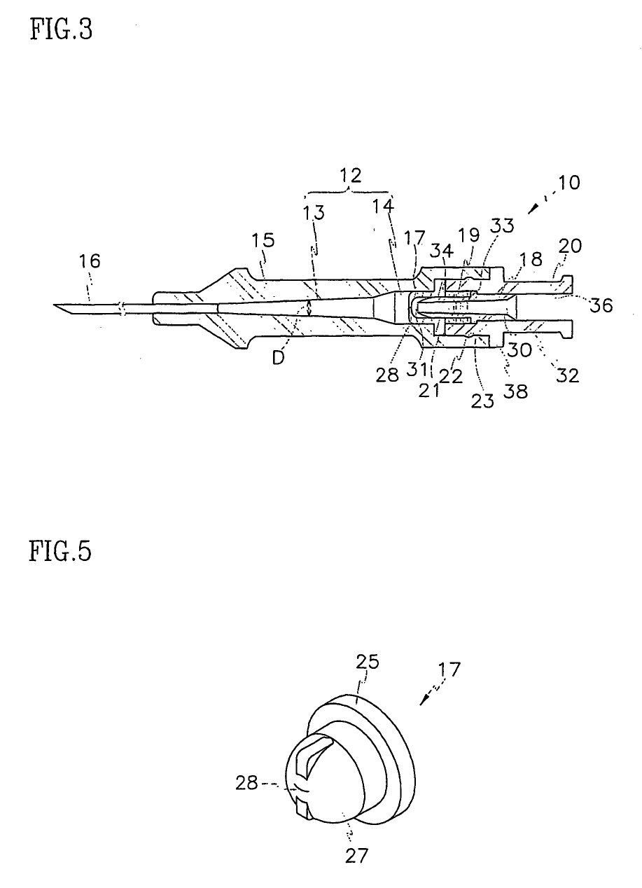

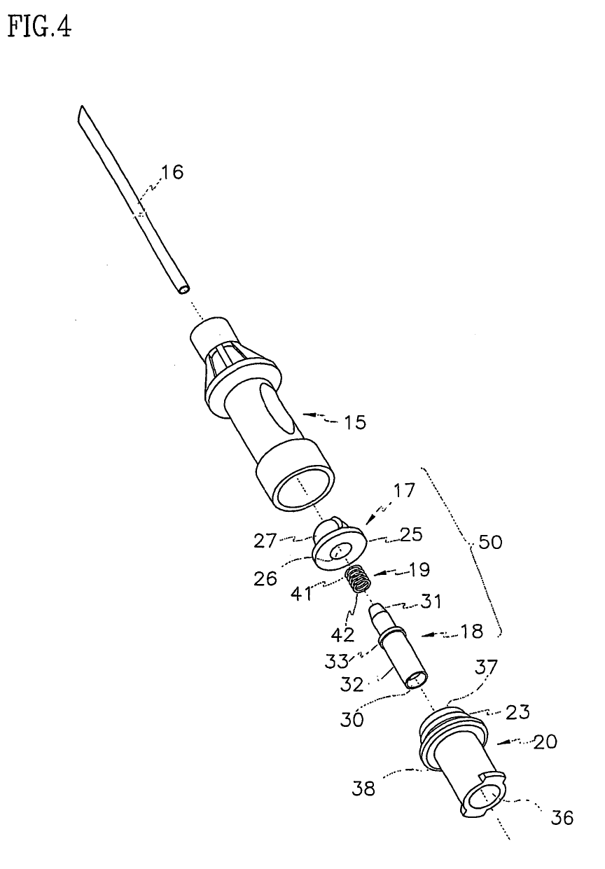

FIG. 2 shows an exploded perspective view of a guide wire insertion device for a catheter according to a preferred embodiment of the present invention, FIG. 3 is a sectional view of a guide wire insertion device depicted in FIG. 2, and FIG. 4 is an exploded perspective view illustrating an internal structure of a guide wire insertion device depicted in FIG. 2.

As shown in the drawings, the inventive guide wire insertion device of a catheter comprises a needle portion 10 having a needle 16 to be inserted into a human body and a syringe 11, through which blood flows in, coupled to the needle portion 10.

The needle portion 10 comprises a hollow body 15, front end of which the needle 16 is coupled, valve means 50 disposed in a hollow portion of the hollow body 15 to check the flow of the blood, and a cap portion 20, in which the syringe 11 is inserted, the cap being integrally mounted on a rear end of the hollow body 15.

Accordingly, the valve means 50 prevents the blood flowed into the body through the needle 16 from being flowing backward. The blood flowed into the body flows into the syringe 11 only when the front end of the syringe 11 penetrates the valve means 50.

The hollow portion 12 of the body 15 comprises a front portion 13 through which the blood flows in, and a rear portion 14 having an inner diameter larger than that D of the front portion.

The inner diameter D of the front portion is designed such that the guide wire 45 can pass there through. The needle 16 has an inner diameter designed such that the guide wire can also pass there through. Accordingly, it is possible to insert the guide wire 45 for guiding the catheter (not shown) into the human body into the human body through the hollow portion 12 of the body 45.

The valve means 50 is provided in the rear portion 14. The valve means 50 comprises a check valve 17 for controlling the flow of the blood, a rod portion mounted on a rear portion of the check valve 17 to open/close the check valve 17, and a spring 19 for biasing the rod rearward to prevent the check valve 17 from being opened.

The check valve 17 is formed of a synthetic resin having a proper elasticity and disposed in the rear portion 14. The check valve 17 has a circular flange 25 provided at its center portion with a circular hole 26 and hooked on a first hook portion 21 formed on the rear portion 14 and a hemispherical portion 27 projected from a surface of the circular flange 25 and facing the needle 16.

The hemispherical portion 27 is provided with a space communicating with the circular hole 26 of the flange 25. In addition, as shown in FIG. 5, the hemispherical portion 27 is further provided at its top with a cut-away portion 28 having a predetermined length. The cut-away portion 28 is closed by the check valve 17 formed of the elastic synthetic resin as far as outer force is not applied. The cut-away portion 28 is opened by being widened when the rod portion 18 pushes the cut-way portion 28. Therefore, when the blood flowed through the needle 16 flows into the check valve 17 through the cut-away portion 28. The cut-way portion may be formed in a straight line-shape or a Y-shape.

The rod portion 18 is formed of a tube provided with a hollow portion 30 and reciprocally mounted in the rear portion 14 of the body 15. One end portion 31 of the rod portion 18 has a diameter less than that of the circular hole 26 such that it can be located in the hemispherical portion 27 over the circular hole 26. The front end of the syringe 11 is inserted into the other end of the rod portion 18 to press the rod portion 18.

Accordingly, when the rod portion 18 is biased forward by the syringe 11, the one end 31 of the rod portion 18 pushes the check valve 17 to open the check valve 17, whereby the blood flows into through the check valve 17 and passes through the rod portion 18.

In addition, an elastic member such as a spring 19 is disposed between the check valve 17 and the rod portion 18. One end 41 of the spring 19 is supported on a side portion the circular flange 25 and the other end of the spring 19 is supported on a side portion of a middle-step 33 of the rod portion 18.

Accordingly, the rod portion 18 pushed forward by the syringe 11 is biased rearward by the spring 19. As a result, when the syringe 11 is biased forward, the rod portion 18 is pushed forward, thereby opening the cut-away portion 28 of the check valve so that the blood flows in. When the biasing force applied to the syringe 11 is released, the rod portion 18 is pushed rearward by the biasing force of the spring, thereby closing the cut-away portion 28 of the check valve 18.

In addition, the cap portion 20 is fitted in the rear portion 14 of the body 15 so as to prevent the check valve 17, the rod portion 18 and the spring 19 from being removed away. The cap portion 20 is provided with a hollow portion 36. The front end of the cap portion 20 is fitted in the rear portion 14 of the body 15. The cap portion 20 is provided at an outer portion of the front end with a hook step 23 coupled to a hook groove formed on an inner circumference of the rear portion 14.

Formed on a middle portion of the cap portion 20 is a second hook step 38 hooked on a side portion of the rear end of the body 15. Accordingly, the cap portion 20 is integrally coupled to the rear end of the body 15, thereby fixing the check valve 17, the spring 19, and the rod portion 18 in the body 15.

The syringe 11 is mounted on the rear end of the cap portion 20 so as to store the blood flowing out through the cap portion 20.

The operation of the above described wire insertion device for the catheter will be described more in detail hereinafter in conjunction with the accompanying drawings.

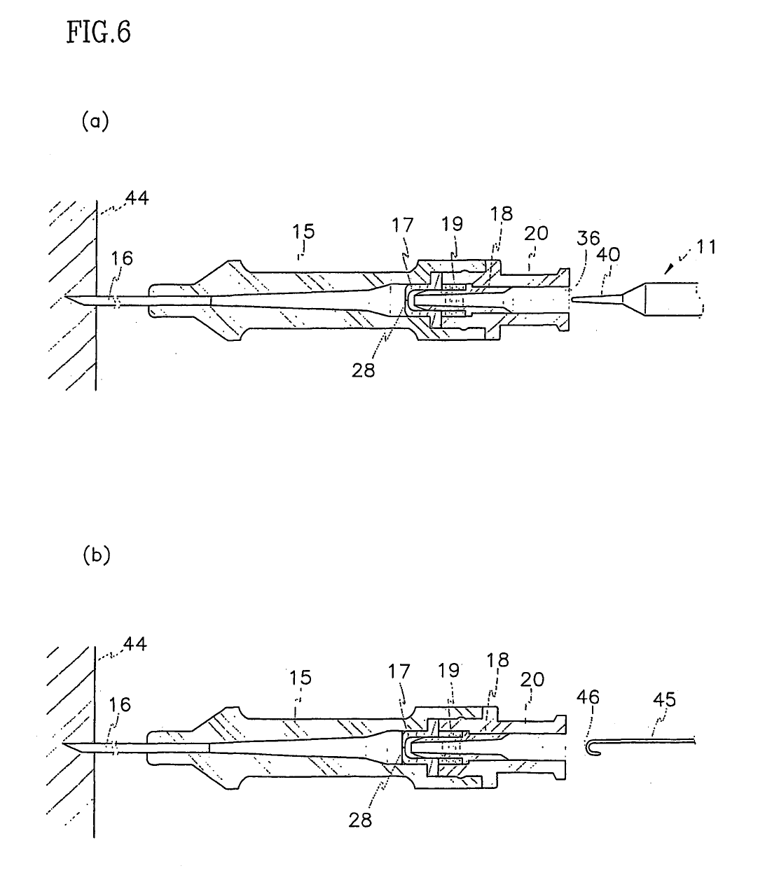

To insert the catheter in the human body, as shown in FIGS. 6(a) and 6(b), the guide wire insertion device is first assembled. That is, the check valve 17, the spring 19 and the rod portion 18 are in this order inserted in the hollow portion of the body 15, after which the cap portion 20 is mounted on the body 15. Then, the syringe 11 is inserted through the hollow portion 36 of the cap portion 20. At this point, since the piston 39 of the syringe 11 is in a state where it is pulled rearward, the inner space of the syringe 11 has pressure lower than the atmosphere pressure.

In this state, when the syringe 11 is pushed forward, the front end 40 of the syringe 11 pushes the rod portion 18 forward. As a result, the rod portion 18 pushes the cut-away portion 28 of the check valve 17 to open the cut-away portion 28. At this point, the spring 19 is in a compressed state.

When the assembling process of the guide wire insertion device for the catheter is completed, the administrator inserts the guide wire insertion device into the human body 44 so that the needle 16 is advanced into the blood vessel.

When the needle 16 is advanced into the blood vessel, since the inner pressure of the blood vessel is higher than that of the syringe 11, the blood is gushed out through the needle 16 and then flows in the syringe 11 through the check valve 17, the rod portion 18 and the cap portion 20.

Accordingly, the administrator can object the blood flowing in the syringe 11, thereby identifying that the needle 16 can exactly inserted into the blood vessel.

After the above, to insert the guide wire 45 into the blood vessel, the syringe 11 is separated from the needle portion 10. At this point, since the force of the syringe 11 which pushes the rod portion 18 forward is released, the rod portion 18 is biased rearward by the elastic force of the spring 19. As a result, the front end 31 of the rod portion 18 can be removed from the cut-away portion 28. Therefore, the cut-away portion is closed to prevent the blood from being gush out from the blood vessel.

In this state, the guide wire 45 is inserted into the needle portion 10 through the cap portion 20. At this point, the front end 46 of the guide wire 45 is advanced into the needle 16 after passing through rod portion 18 and the cut-away portion of the check valve 17, and then inserted into the blood vessel.

By the above-described procedure, the guide wire can be inserted into a desired location of the blood vessel.

After the guide wire 45 is inserted into the blood vessel, the guide wire insertion device is removed from the human body and the catheter is inserted in the human body to treat the human body.

As described above, the guide wire insertion device for a catheter of the present invention has an advantage that it can prevent blood from being gush out when a needle is inserted into a blood vessel.

In addition, when the guide wire is advanced into the human body after the guide wire insertion device for the catheter is inserted into the human body, since the wire is directly inserted into the needle portion after the syringe is removed, the whole length of the device can be shortened, making it easy for the administrator to operate the device.

Although the embodiments of the present invention have been explained in detail above, the present invention can be modified in a variety of ways without departing from the spirit and scope of the invention.

What is claimed is:

1. A guide wire insertion device for a catheter, comprising:
a needle portion provided with a hollow portion, the needle portion having a needle mounted on a front end;
valve means mounted in the hollow portion of the needle portion to control flow of blood;
a cap portion mounted on a rear end of the hollow portion of the needle portion to fix the valve means in the needle portion; and
a syringe for absorbing the blood through the needle portion by penetrating the valve means.

2. A guide wire insertion device of claim 1 wherein the valve means comprises a check valve for controlling the flow of the blood, a rod portion mounted on a rear portion of the check valve to open/close the check valve, and a spring for biasing the rod rearward to prevent the check valve from being opened when the syringe is removed by pushing the rod portion rearward.

3. A guide wire insertion device of claim 2 wherein the check valve comprises a circular flange having a central circular hole and a hemispherical portion projected from a surface of the circular flange and facing the needle, the hemispherical portion being provided at its top with a cut-away portion and a hollow portion communicating with the circular hole of the circular flange.

4. A guide wire insertion device of claim 3 wherein the check valve is designed such that the cut-away portion is elastically closed by self-elastic force.

5. A guide wire insertion device of claim 5 wherein the rod portion is formed in a hollow tube, one end of the rod portion has a diameter less than that of the circular hole such that the one end can be advanced into the hemispherical portion through the circular hole, wherein when the syringe is pushed forward, a front end of the syringe pushes the rod portion forward to open the cut-away portion.

6. A guide wire insertion device of claim 5 wherein one end of the spring is supported on the circular flange of the check valve and the other end of the spring is supported on a middle step formed on a middle portion of the rod portion.

7. A guide wire insertion device of claim 3 wherein the cap portion is provided with a hollow portion in which the rod portion is reciprocally mounted, the cap portion being fixed on the needle portion by a hook step formed on a front end of the cap portion and coupled to a hook groove formed on an inner circumference of the needle portion.
